(19) United States
(12) Patent Application Publication
(10) Pub. No.: US 2008/0312433 A1
(43) Pub. Date: Dec. 18, 2008

(54) Novel process to Form-I of olanzapine

(75) Inventors: Daqing Che, Brantford (CA); Kiran Kumar Kothakonda, Brantford (CA); Cameron L. McPhail, Brantford (CA); Bhaskar Reddy Guntoori, Brantford (CA)

Correspondence Address: IVOR M. HUGHES, BARRISTER & SOLICITOR,;PATENT & TRADEMARK AGENTS, 175 COMMERCE VALLEY DRIVE WEST, SUITE 200, THORNHILL, ON, L3T 7P6, CA

(73) Assignee: Apotex Pharmachem Inc.

(21) Appl. No.: 11/976,944

(22) Filed: Oct. 30, 2007

(30) Foreign Application Priority Data
Jun. 14, 2007 (CA) 2,591,644

(51) Int. Cl. C07D 495/04

(52) U.S. Cl. 540/557

(57) ABSTRACT

A process for obtaining crystalline Form-I olanzapine comprising the following:
a) dissolving crude olanzapine in a solvent to form a solution,
b) optionally drying by azeotropic distillation to remove water,
c) precipitating by adding the solution of step (a) to an antisolvent, and
d) isolating the precipitated crystalline Form-I olanzapine by filtration and drying at ambient temperature.

### FIELD OF THE INVENTION

Novel and high-yielding processes to prepare substantially pure Form-I of olanzapine are disclosed whereby crude olanzapine is dissolved in a solvent and the resulting solution is added to an antisolvent (such as heptanes or methyl tert-butyl ether).

### BACKGROUND OF THE INVENTION

Olanzapine (1,2-methyl-4-(4-methyl-1-piperazinyl)-10H-thieno[2,3-b][1,5]benzo-diazepine) is a second generation anti-psychotic drug marketed as Zyprexa® by Eli Lilly and Company. It is useful for the treatment of disorders such as schizophrenia, bipolar disorder, psychotic depression and Tourette syndrome. This pharmaceutical acts as an antagonist on 5-HT₂ serotonin receptors as well as the D₁/D₂ dopamine receptors and also exhibits anticholinergic and antimuscarine properties.

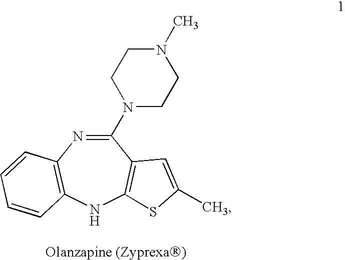

The synthesis of olanzapine and its application as a pharmaceutical are reported, for example, in U.S. Pat. No. 5,229,382 (U.S. Pat. No. '382). Subsequent to this, U.S. Pat. No. 5,736,541 disclosed a more stable anhydrate Form-I crystal form of olanzapine in addition to the meta-stable Form-I. It also indicated that the Form-I polymorph was produced according to the procedures of U.S. Pat. No. '382. Obtaining the olanzapine Form-I polymorph is challenging due to the fact that, purportedly, it readily discolours on exposure to air, and therefore is unsuitable for commercial use.

Since then, numerous olanzapine polymorph patents have been issued covering other solvated, amorphous, crystalline anhydrate and hydrated forms. For example, U.S. Pat. No. 6,348,458 teaches three other crystalline polymorphic forms, namely Forms III, IV and V. WO 03/091260 teaches a Form VI and WO 2006/102176 teaches a Form X. U.S. Pat. No. 6,020,487 reports three dihydrate polymorphs, Forms D, B and E. Likewise, U.S. Pat. No. 5,631,250, U.S. Pat. No. 5,703,232, US 2006/0223794 and WO 1999/16313 teach various solvated forms of olanzapine. WO 2004/113346 teaches an amorphous form of olanzapine. An article in Crystal Growth and Design, 2003, Vol. 3, No. 6, pp. 897-907 by Reutzel-Edens et al. discusses the polymorphism of olanzapine.

In terms of the specific Form-I polymorph, U.S. Pat. No. 6,432,943 describes a method of obtaining substantially pure crystalline Form-I (incorrectly identified as Form-II) using commercially undesirable methylene chloride as the crystallizing solvent. It also discloses methods of obtaining the stable polymorph Form-II (incorrectly identified as Form-I in the patent) using solvents such as acetone, ethyl acetate and tert-butyl alcohol. Subsequently, other methods were reported in other patents for obtaining polymorphic Form-I; however all of these methods use commercially undesirable methylene chloride at some point in the process.

Other patents and patent applications disclose processes to prepare substantially pure polymorphic Form-I. US 2005/0272720 A1 teaches a process for obtaining Form-I via the industrially impractical high temperature decomposition of the acetate salt. WO 2006/027800 A1 teaches a method of providing anhydrous Form-I using water miscible solvents such as dimethyl sulfoxide, N,N-dimethylformamide, tetrahydrofuran and methanol, which also require further high temperature drying to obtain anhydrous material. The main disadvantage with these methods is that higher temperatures can result in conversion of desirable Form-I olanzapine to Form-II olanzapine.

Finally, WO 2007/009788 A1 teaches a process for making Form-I by reducing the pressure of a supercritical fluid composition comprising carbon dioxide and olanzapine.

It is therefore an object of the invention to provide an industrially viable process for the production of Form-I olanzapine.

It is a further object of the invention to employ industrially acceptable low boiling solvents to produce Form-I olanzapine.

Further and other objects of the invention will become apparent to those skilled in the art when considering the following summary of the invention and the more detailed description of the embodiments of the invention described herein.

### SUMMARY AND DETAILED DESCRIPTION OF THE INVENTION

According to one aspect of the invention, there is provided a process for obtaining crystalline Form-I olanzapine comprising the following:
a) dissolving crude olanzapine in a solvent,
b) optionally drying by azeotropic distillation to remove water,
c) precipitating by adding the solution to an antisolvent, and
d) isolating the precipitated crystalline Form-I olanzapine by filtration and drying at ambient temperature.

According to another aspect of the invention, a process is provided for obtaining a substantially pure polymorphic Form-I of olanzapine from crude olanzapine. This method uses industrially acceptable class-3 solvents as listed in the ICH Q3C (R3) guidelines such as, for example, 2-butanol, heptanes, methyl tert-butyl ether (MTBE) and methyl iso-butyl ketone (MIBK). 2-Butanol is particularly preferred because it has many characteristics which make it highly attractive for use as a solvent for the industrial preparation of pharmaceuticals, including olanzapine, such as it is inexpensive, has low-toxicity and is readily available.

Further, according to another aspect of the invention a robust and high-yielding process is provided for obtaining crystalline Form-I olanzapine using, for example, 2-butanol (2-butyl alcohol, sec-butanol) and an antisolvent, for example, heptanes or MTBE, via a reverse-addition technique. The crystalline Form-I olanzapine produced by this process has the characteristic x-ray diffraction pattern approximate d-values (in angstroms) of: 9.96, 8.58, 8.25, 6.90, 6.38, 5.95, 5.60, 4.98, 4.84, 4.77, 4.72, 4.63, 4.54, 4.47, 4.25, 4.10, 3.83, 3.76, 3.70, 3.59, 3.51, 3.35, 3.29, 3.25, 3.22, 3.19, 3.12, 3.06, 2.96, 2.89, 2.82, 2.76, 2.71, 2.66, 2.59, 2.58, 2.49, 2.47, 2.43, 2.39, 2.34.

In a preferred embodiment of the invention, crude olanzapine can be dissolved in a volume of a solvent, such as 2-butanol, at a temperature from about 50 to about 105° C. to form a solution. The volume of solvent used can be 2 to 10 volumes, preferably 2 to 8 volumes and most preferably 2 to 6 volumes. The term ‘volumes’ used above and throughout this patent represents the volume of solvent (in L) relative to the weight of olanzapine (in kg). For instance, 2 volumes corresponds to 2 L of solvent per kg of olanzapine.

Optionally, a certain amount of the solvent can be removed by distillation to produce an anhydrous solution. An anhydrous solution is defined in this case as a solution whose Karl Fisher (KF) value ranges from about 0.05-1.0%. To achieve this KF value, the volume of solvent distilled is typically between about 0.05 to 5 volumes, and most preferably 1 to 3 volumes.

The temperature of the solution can be between about 25 to 110° C., preferably 50 to 105° C. and most preferably 65-103° C. Optionally, the solution can be clarified by, for instance, filtering through a Buchner funnel packed with a filtering medium, such as Celite®.

The solution can then be added to an antisolvent. This type of addition is known in the art as a reverse addition. The volume of antisolvent can be between about 1 to 15 volumes, preferably 2 to 12 volumes and most preferably 3 to 10 volumes. Suitable antisolvents are selected from the group consisting of C₅ to C₁₀ hydrocarbons such as heptanes, C₄ to C₈ alkyl ethers such as MTBE, C₃ to C₇ alkyl ketones such as methyl iso-butyl ketone (MIBK), or mixtures thereof. Most preferably, the antisolvent is heptanes or MTBE, or mixtures thereof.

The addition and precipitation temperature ranges from about −20° C. to about 20° C., preferably from about −10° C. to about 15° C. and most preferably from about −5 to about 10° C. Optionally, pure seed crystals of Form-I olanzapine can be added to the antisolvent before or after addition of the 2-butanol solution. The amount of seed crystals ranges from about 0.01 to 10% weight-by-weight (w/w) of crude olanzapine, preferably 0.05 to 5% w/w and most preferably 0.1 to 2% w/w

The following non-limiting examples further illustrate the manner of carrying out the inventive process described herein.

### EXAMPLE 1

Crude olanzapine (10 g) was charged to a three necked flask along with 40 mL of 2-butanol and heated to dissolve. 10 mL of solvent was removed by atmospheric distillation and the hot solution was clarified and then added to MTBE (30 mL) (<5 min) and the temperature was maintained between −5 and 5° C. for precipitation. After complete precipitation, the product was isolated by filtration and dried at ambient temperature in a vacuum oven to obtain 7 g of pure Form-I.

### EXAMPLE 2

Olanzapine (10 g) was charged to a three necked flask along with 40 mL of 2-butanol and heated to dissolve. 10 mL was removed by atmospheric distillation and the hot saturated solution was added to 50 mL of MTBE at −15° C. Form-I seeds (1% relative to the weight of the olanzapine) were added and the temperature was maintained between −5° C. and 5° C. for precipitation. After complete precipitation, the product was isolated by Buchner filtration and dried in a vacuum oven at ambient temperature to provide 7 g of pure Form-I olanzapine.

### EXAMPLE 3

Olanzapine (10 g) was charged to a three necked flask along with 40 mL of 2-butanol and the mixture was heated to dissolve. 10 mL of 2-butanol was removed by distillation and 10 mL of the hot saturated solution was added to MTBE (50 mL) (at −15° C.) followed by Form-I olanzapine seeds [0.25% (w/w)] and the temperature was maintained between −5° C. and 5° C. for precipitation. The remaining 20 mL were added and another portion of seeds [0.25% (w/w)] was added. After complete precipitation, the precipitated Form-I olanzapine was isolated by filtration and dried at ambient temperature in a vacuum oven to provide 7 g.

### EXAMPLE 4

Olanzapine (100 g) was charged to a three necked flask along with 600 mL of 2-butanol and the mixture was heated to dissolve. The solution was clarified by Buchner filtration and 200 mL of 2-butanol were removed by distillation. The hot saturated solution was added to MTBE (400 mL) at −15° C. followed by Form-I olanzapine seeds [0.2% (w/w)] and the temperature was maintained between −5° C. and 5° C. for precipitation. After complete precipitation, the precipitated Form-I olanzapine was isolated by filtration and dried at ambient temperature in a vacuum oven to provide 81 g.

### EXAMPLE 5

Olanzapine (10 g) was charged to a three necked flask, followed by 60 mL of 2-butanol and this mixture was heated to dissolution. It was filtered into other flask and refluxed. 20 mL of 2-butanol were removed by distillation and the hot saturated solution was added to 40 mL of heptanes and the temperature was maintained between −5° C. and 5° C. for precipitation. After complete precipitation, the product was isolated by filtration and dried at ambient temperature in a vacuum oven to provide 9 g Form-I olanzapine.

### EXAMPLE 6

Olanzapine (100 g) was charged to a three necked flask, followed by 600 mL of 2-butanol and the mixture was heated to dissolution. The solution was then clarified by filtration into a flask and refluxed. 200 mL of 2-butanol were removed by atmospheric distillation, the hot saturated solution was added to heptanes (400 mL) at −15° C. containing 0.1% Form-I seeds (w/w) and thereafter the temperature was maintained between −5° C. and 15° C. for precipitation. After complete precipitation, the solution was filtered and the isolated product was dried in a vacuum oven at ambient temperature to obtain 86 g of pure Form-I olanzapine.

As many changes can be made to the invention without departing from the scope of the invention, it is intended that all material contained herein be interpreted as illustrative of the invention and not in a limiting sense.

1. A process to prepare Form-I olanzapine comprising the steps of:
i. dissolving crude olanzapine in a solvent selected from 2-butanol, heptanes, methyl tert-butyl ether (MTBE) and methyl iso-butyl ketone (MIBK) to form a solution,
ii. optional filtration of the product of step (i),
iii. optional distillation,
iv. precipitating by adding the solution to an antisolvent selected from C₅ to C₁₀ hydrocarbons, C₄ to C₈ alkyl ethers, C₃ to C₇ alkyl ketones, or mixtures thereof,
v. isolating of Form-I olanzapine by filtration, and
vi. optionally drying the Form-I olanzapine.

2. The process of claim 1 wherein the antisolvent is further selected from the group consisting of methyl tert-butyl ether, heptanes, methyl iso-butyl ketone, or mixtures thereof.

3. The process of claim 1 or 2 wherein the solvent is 2-butanol.

4. The process of claim 3 wherein the amount of 2-butanol is 2 to 10 volumes.

5. The process of claim 3 wherein the amount of 2-butanol is 2 to 8 volumes.

6. The process of claim 3 wherein the amount of 2-butanol is 2 to 6 volumes.

7. The process of claim 3 wherein the solution is maintained at a temperature of between 25 to 110° C.

8. The process of claim 3 wherein the solution is maintained at a temperature of between 50 to 105° C.

9. The process of claim 3 wherein the solution is maintained at a temperature of between 65-103° C.

10. The process of claim 3 wherein 0.05 to 5 volumes of 2-butanol is removed by distillation.

11. The process of claim 3 wherein 1 to 3 volumes of 2-butanol is removed by distillation.

12. The process of claim 3 wherein the amount of antisolvent is between 1 to 15 volumes.

13. The process of claim 3 wherein the amount of antisolvent is between 2 to 12 volumes.

14. The process of claim 3 wherein the amount of antisolvent is between 3 to 10 volumes.

15. The process of claim 3 wherein the addition and precipitation temperature ranges from about −20° C. to about 20° C.

16. The process according to claim 3 wherein step (iv) is accomplished in the presence of Form-I seeds.

17. The process according to claim 3 wherein step (iv) is accomplished in the presence of Form-I seeds ranging from 0.01 to 10% (w/w) relative to the weight of olanzapine.

18. The process according to claim 3 wherein step (iv) is accomplished in the presence of Form-I seeds ranging from 0.05 to 5% (w/w) relative to the weight of olanzapine.

19. The process according to claim 3 wherein step (iv) is accomplished in the presence of Form-I seeds ranging from 0.1 to 2% (w/w) relative to the weight of olanzapine.

20. The process according to claim 3 wherein the Form-I olanzapine has the characteristic x-ray diffraction pattern approximate d-values (in angstroms) of: 9.96, 8.58, 8.25, 6.90, 6.38, 5.95, 5.60, 4.98, 4.84, 4.77, 4.72, 4.63, 4.54, 4.47, 4.25, 4.10, 3.83, 3.76, 3.70, 3.59, 3.51, 3.35, 3.29, 3.25, 3.22, 3.19, 3.12, 3.06, 2.96, 2.89, 2.82, 2.76, 2.71, 2.66, 2.59, 2.58, 2.49, 2.47, 2.43, 2.39, 2.34.
